(19) United States
(12) Patent Application Publication
(10) Pub. No.: US 2008/0261823 A1
(43) Pub. Date: Oct. 23, 2008

(54) Fluorescent Nucleoside Analogs That Mimic Naturally Occurring Nucleosides

(76) Inventor: Yitzhak Tor, San Diego, CA (US)

Correspondence Address: DLA PIPER US LLP, 4365 EXECUTIVE DRIVE, SUITE 1100, SAN DIEGO, CA, 92121-2133, US

(21) Appl. No.: 11/792,968

(22) PCT Filed: Dec. 9, 2005
(86) PCT No.: PCT/US05/44549

§ 371 Date: Jan. 10, 2008

Related U.S. Application Data
(60) Provisional application No. 60/635,052, filed on Dec. 10, 2004.

(51) Int. Cl. C40B 30/04; C07H 19/073; C07H 21/04; C07H 19/23; C07H 19/24; G01N 33/58

(52) U.S. Cl. 506/9; 536/26.8; 536/24.5; 536/26.7; 536/23.1; 436/94; 536/27.1

(57) ABSTRACT

The present invention provides fluorescent nucleoside analogs with conjugated membered heterocycles, including furan and thiophene. The fluorescent nucleoside analogs maintain structural similarity to naturally occurring nucleoside bases, mimicking shape, size, hybridization, and recognition properties. Incorporation of the fluorescent cyclic compounds confers specific photophysical characteristics including a bathochromic (red) shift of the absorption spectrum to minimize absorption overlap with naturally occurring nucleoside bases, and a shift to the long emission wavelength in the visible range. The invention also provides for various methods of synthesizing the fluorescent nucleoside analogs and incorporating the fluorescent analogs in DNA, RNA, or oligomer synthesis. Further, methods of detecting the fluorescent nucleoside analogs in an oligonucleotide or oligomer are provided. The subject compounds are useful as probes in the study of the structure and dynamics of nucleic acids and their complexes with proteins.

### RELATED APPLICATION

This application claims priority under 35 U.S.C. §119(e) to U.S. Provisional Application 60/635,052 filed Dec. 10, 2004, herein incorporated by reference in its entirety.

### STATEMENT OF GOVERNMENT SUPPORT

This invention was made in part with government support under Grant No. GM69773 awarded by the National Institutes of Health. The government has certain rights in this invention.

### FIELD OF THE INVENTION

This invention relates to fluorescent nucleoside analogs as probes for nucleic acid structure, dynamics, and function as well as sequence and lesion analysis.

### BACKGROUND INFORMATION

Fluorescence methods are extremely widespread in chemistry and biology. The methods give useful information on sequence, structure, distance, orientation, complexation, location for biomolecules, and measurements of dynamics and kinetics. As a result, many strategies for fluorescence labeling of biomolecules, including nucleic acids, have been developed.

For example, in the case of DNA, a convenient and useful method for fluorescence labeling is to add a fluorescent moiety during the DNA synthesis itself. However, the majority of labels commonly used during DNA synthesis are attached to the DNA as tethers that are often 5 to 11 or more atoms long. These tethers can be problematic, for example, the tethers make the location of the dye difficult to determine precisely, interfere with DNA-protein interactions, etc.

Unfortunately, fluorescent nucleosides that mimic naturally occurring nucleoside bases structurally and chemically are scarce. Thus, a need exists for fluorescent nucleosides which are structurally and chemically similar to naturally occurring nucleosides. The present invention provides nucleoside analogs with improved photophysical characteristics and the subject nucleoside analogs are more generally useful in biophysical and diagnostics applications.

### SUMMARY OF THE INVENTION

The present invention relates to fluorescent nucleoside analogs containing conjugated 5-membered heterocycles. In particular, the 5-membered heterocycles confer improved photophysical properties to the analogs.

The present invention also relates to fluorescent analogs containing conjugated 5-membered heterocycles which maintain the structural similarity to that of naturally occurring nucleoside bases (i.e., purines and pyrimidines), including substantially similar shape, sizes, hybridization and recognition capabilities. Further the fluorescent analogs of the invention have advantageous photophysical characteristics over that of the naturally occurring nucleoside bases, including emission spectrum in the longer wavelengths (i.e., towards the visible range), bathochromatic (red) shifted absorption spectrum such that there is minimization of overlap with the naturally occurring nucleoside bases.

In one embodiment, a compound is provided having the general formula (I):

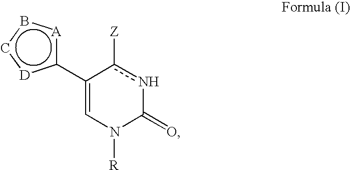

where dashed lines represent optional double bonds, A and B are, independently, —CH═, —O—, or —S—, and A and B are different where ever they appear, C and D are, independently, —N═ or —CH═, and Z is —NH₂ or ═O, with the proviso that when A is —O— or —S—, B is not —O— or —S—, and when C is —N═, D is not —N═, R is —H or a glycal having the general formula (II) a or (II)b

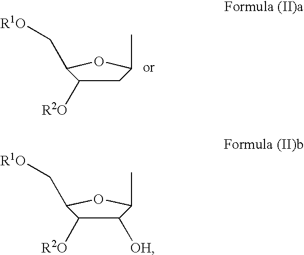

### R¹ is —H, —PO₃, or

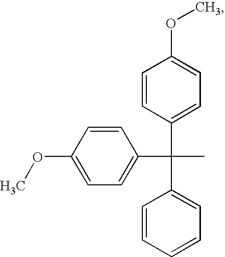

### and R² is —H, —PO₃, or

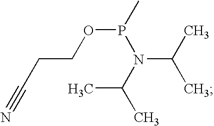

or salts thereof.

In one aspect, A is —O—, B, C, and D are —C═, Z is ═O, and R is

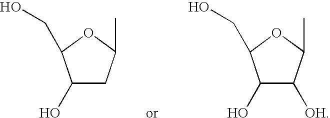

In another aspect, A is —S—, B, C, and D are —C═, Z is ═O, and R is

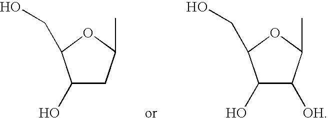

In a further aspect, A is —O—, D is —N═, Z is ═O, and R is

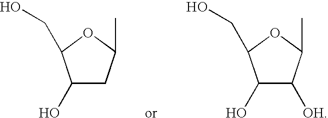

In another aspect, A is —S—, D is —N═, Z is ═O, and R is

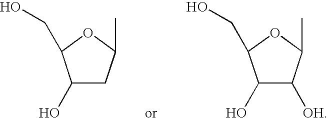

In another embodiment, a synthetic oligonucleotide is provided including at least one compound of having the general formula (I), where the synthetic oligonucleotide substantially hybridizes to a complementary naturally occurring polynucleotide or oligonucleotide, including where the synthetic oligonucleotide, naturally occurring polynucleotide, and naturally occurring oligonucleotide comprises DNA or RNA.

In one embodiment, a compound is provided having the general formula (III):

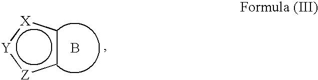

where each of X and Y is, independently, —O—, —S—, or —CH═, and X and Y are different where ever they appear, with the proviso that when X is —O— or —S—, Y is not —O— or —S—, Z is selected from —CH═, —N═, or —CR¹═, and ring B is selected from:

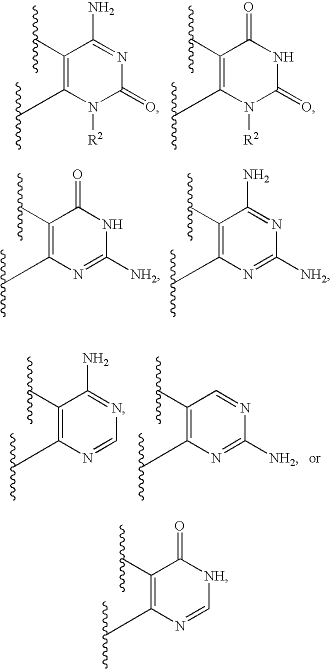

where R¹ and R² are each the same or different, where ever they appear, and each is selected from —H or a glycal having the general formula (II)a or (II)b:

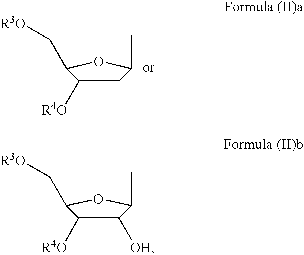

### R³ is —H, —PO₃, or:

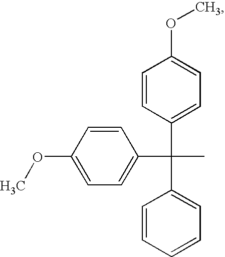

### and R⁴ is —H, —PO₃, or:

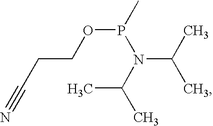

with the proviso that when R¹ is a glycal, R² is not a glycal;
or salts thereof.

In one aspect, X is —O—, Z is —CR¹═, ring B is

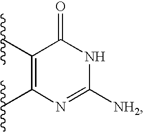

and R¹ is

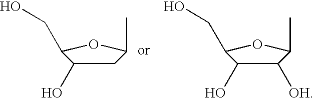

In another aspect, X is —S—, Z is —CR¹═, ring B is

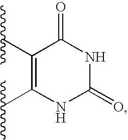

### and R¹ is

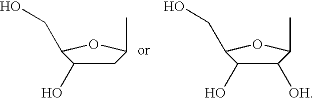

In a further aspect, X is —S—, Z is —CH═, ring B is

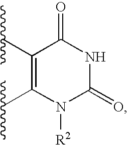

### and R² is

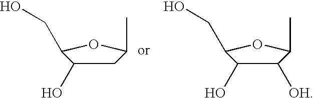

In another embodiment, a synthetic oligonucleotide is provided including at least one compound having the general formula (III), where the synthetic oligonucleotide substantially hybridizes to a complementary naturally occurring polynucleotide or oligonucleotide, including where the synthetic oligonucleotide, naturally occurring polynucleotide, and naturally occurring oligonucleotide comprises DNA or RNA.

In one embodiment, a compound is disclosed having the general formula (IV):

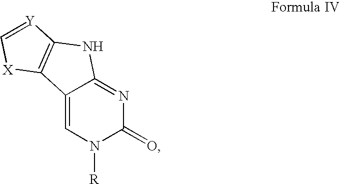

where, X and Y are, independently, —CH═, or —O—, and X and Y are different where ever they appear, with the proviso that when X is —O—, Y is not —O—, R is —H or a glycal having the general formula (II)a or (II)b:

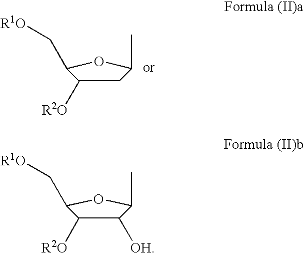

### R¹ is —H, —PO₃, or

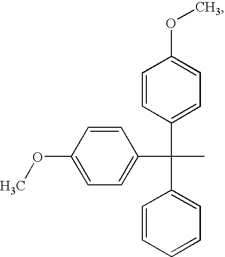

### and R² is —H, —PO₃, or

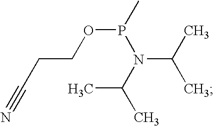

or salts thereof.

In one embodiment, a compound is disclosed having the general formula (V):

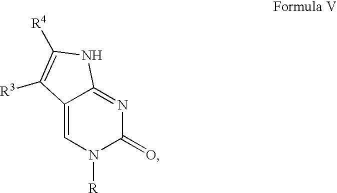

where R is —H or a glycal having the general formula (II)a or (II)b:

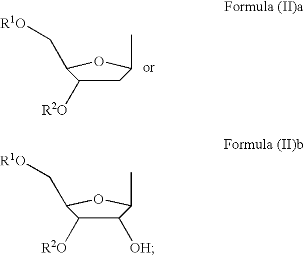

### R¹ is —H, —PO₃, or

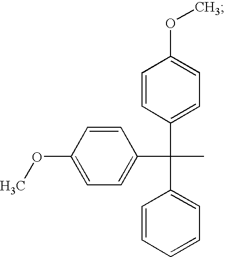

### R² is —H, —PO₃, or

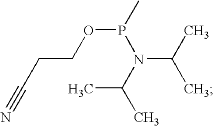

R³ and R⁴ are each independently —H or a furan having the general formula (VI):

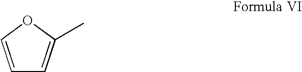

where each is different where ever they appear with the proviso that
when R³ is Formula (VI), R⁴ is not Formula (VI);
   or salts thereof.

In one embodiment, kits are described, including at least one compound of the general formula (I) or (III) or an oligonucleotide comprising the at least one compound of the general formula (I) or (II), a container, and directions for using the at least one compound or oligonucleotide. In a related aspect, the at least one compound is a phosphoramidite derivative.

In another embodiment, a method of synthesizing 5-modified pyrimidine analogs is provided comprising admixing 5-iodo-2′-deoxyuridine or 3′,5′-diTol-Iodo-dU and the corresponding stannylated heterocycles in the presence of palladium, protecting the 5′-hydroxyl with 4,4′-dimethoxytrityl chloride, and phosphitylating the unprotected 3′-hydroxyl.

In one embodiment, a method is disclosed for synthetically preparing a fluorescently labeled oligonucleotide including incorporating at least one compound of the general formula (I) or (III) into a DNA or RNA chain. In a related aspect, the method further comprises admixing the at least one compound with a growing DNA or RNA chain, where the at least one compound is a phosphoramidite derivative, including synthesis on a solid phase.

In another embodiment, a method is disclosed for detecting a target nucleic acid in a sample including, contacting the sample with one or more oligonucleotides having at least one compound of the general formula (I) or (III) incorporated therein, for a time and under conditions sufficient to allow hybridization to occur between the target nucleic acid and the oligonucleotides, separating non-hybridized oligonucleotides, exciting the hybridized oligonucleotides, and detecting fluorescence produced by complexes formed between the oligonucleotides and the target nucleic acid, where detecting fluorescence correlates with the presence of the target nucleic acid.

The invention also provides methods for synthesizing the fluorescent nucleoside analogs that maintain structural similarity to naturally occurring nucleoside bases and with conjugated fluorescent 5-membered heterocyles. These methods include cross-coupling the heterocyle to the naturally occurring nucleoside, N-glycosylation the heterocyle to the naturally occurring nucleoside, and C-glycosylation the heterocyle to the naturally occurring nucleoside. Other methods of synthesizing the fluorescent analogs which is known or standard in the art, or which will become known or standard in the art is anticipated and within the scope of the present invention.

The invention also provides methods of preparing fluorescently labeled nucleic acid molecules incorporating at least one fluorescent nucleoside analog of the present invention, for example into an RNA or DNA molecule under conditions sufficient to incorporate the fluorescent nucleoside analog. Similarly, the invention provides for nucleotide analogs comprising one or more fluorescent nucleoside analogs of the present invention.

The invention also provides methods of detecting a target nucleic acid molecule in a sample to be tested by contacting the target nucleic acid with a nucleic acid probe containing at least fluorescent nucleoside analog for time and under conditions sufficient to permit hybridization between the target nucleic acid molecule and the fluorescent probe and detecting the hybridization.

The invention also provides for an array containing multiple solid supports and multiple locations on a solid support where each support or location has attached an oligomer containing the fluorescent nucleoside analogs.

### BRIEF DESCRIPTION OF THE FIGURES

FIG. 1 shows fluorescent nucleotides divided into subgroups: a) furo- and thieno-pyrimidines as fluorescent purine analogs (structures A-F); b) 5-modified pyrimidines as fluorescent pyrimidine molecules (structures G-L); and c) furo-, thieno-, and oxazolo-pyrimidines as fluorescent pyrimidine analogs (structures M-P).

FIG. 2 shows chemical structures of modified nucleosides (structures 1-5).

FIG. 3 shows selected synthetic routes utilized for the synthesis of modified nucleosides. FIG. 3A: Synthesis of a modified N-nucleoside 7; and FIG. 3B: Synthesis of a C-nucleoside 6 where the brominated heterocycle is cross-coupled to a glycal.

FIG. 4 is a graph showing the emission spectra of nucleoside 1 in various solvents ranging from water (most polar) to diethyl ether (least polar).

FIG. 5 is a graph illustrating the hyperchromism (enhanced emission) and bathochromic (red) shift displayed by nucleoside 1 and its sensitivity (“responsiveness”) to environmental changes.

### DETAILED DESCRIPTION OF THE INVENTION

Fluorescent nucleoside analogs with high emission quantum efficiency and long emission wavelength are usually associated with significant structural and chemical modifications when compared to their natural counterparts. The major challenge in this field is, therefore, to design nucleoside analogs with “optimal” photophysical characteristics (e.g., red-shifted absorption and emission spectra and highest possible emission quantum yield) while maintaining high structural homology to the naturally occurring nucleoside bases.

Nucleotides or oligonucleotides or oligomers of the present invention, comprising naturally occurring nucleotides and phosphodiester bonds can be chemically synthesized or can be produced using recombinant DNA methods, using an appropriate polynucleotide as a template. In comparison, an oligonucleotide comprising nucleotide analogs or covalent bonds other than phosphodiester bonds generally will be chemically synthesized, although an enzyme such as T7 polymerase can incorporate certain types of nucleotide analogs into an oligonucleotide and, therefore, can be used to produce such an oligonucleotide recombinant from an appropriate template (Jellinek et al., supra, 1995).

The present invention also provides methods of synthesizing fluorescent analogs containing conjugated 5-membered heterocycles (e.g., furan and thiophene). The present invention also provides fluorescent nucleoside analog compositions, which when incorporated into a nucleoside framework, confer advantageous and beneficial photophysical characteristics.

In one embodiment, a compound is disclosed having the general formula (I):

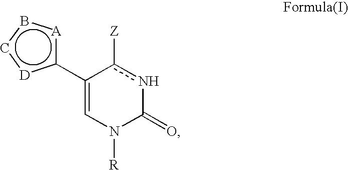

where dashed lines represent optional double bonds, A and B are, independently, —CH═, —O—, or —S—, and A and B are different where ever they appear, C and D are, independently, —N— or —CH═, and Z is —NH₂ or ═O, with the proviso that when A is —O— or —S—, B is not —O— or —S—, and when C is —N═, D is not —N═, R is —H or a glycal having the general formula (II)a or (II)b:

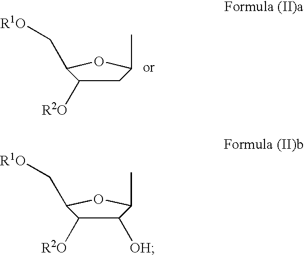

### R¹ is —H, —PO₃, or

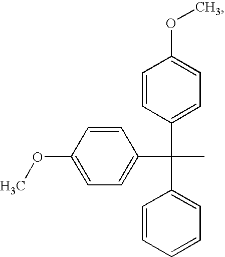

### and R² is —H, —PO₃, or

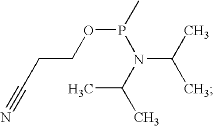

or salts thereof.

In one aspect, A is —O—, B, C, and D are —C═, Z is ═O, and R is

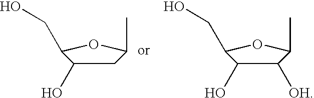

In another aspect, A is —S—, B, C, and D are —C═, Z is ═O, and R is

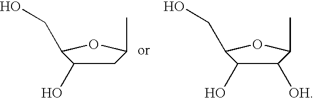

In a further aspect, A is —O—, D is —N═, Z is ═O, and R is

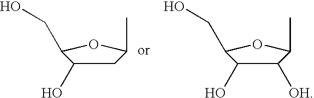

In another aspect, A is —S—, D is —N═, Z is ═O, and R is

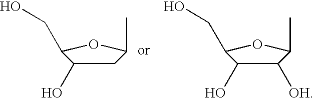

In one aspect, compounds include, but are not limited to, 5-(1,3-oxazol-2-yl)pyrimidine-2,4(1H, 3H)-dione; 5-(2-furyl)pyrimidine-2,4(1H, 3H)-dione; 4-amino-5-(1,3-oxazol-2-yl)pyrimidin-2(1H)-one; 4-amino-5-(2-furyl)pyrimidin-2(1H)-one; 4-amino-5-(2-furyl)pyrimidin-2-(1H)-one; 5-(1,3-oxazol-5-yl)pyrimidine-2,4(1H, 3H)-dione; 5-(2-furyl)pyrimidine-2,4(1H, 3H)-dione; 4-amino-5-(1,3-oxazol-5-yl)pyrimidin-2(1H)-one; 4-amino-5-(2-furyl)pyrimidin-2(1H)-one; 5-(1,3-oxazol-4-yl)pyrimidine-2,4(1H, 3H)-dione; 5-(3-furyl)pyrimidine-2,4(1H, 3H)-dione; 4-amino-5-(1,3-oxazol-4-yl)pyrimidin-2(1H)-one; 4-amino-5-(3-furyl)pyrimidin-2(1H)-one; 5-(1,3-thiazol-2-yl)pyrimidine-2,4(1H, 3H)-dione; 5-(2-thienyl)pyrimidine-2,4(1H, 3H)-dione; 4-amino-5-(1,3-thiazol-2-yl)pyrimidin-2(1H)-one; 4-amino-5-(2-thienyl)pyrimidin-2(1H)-one; 5-(1,3-thiazol-5-yl)pyrimidine-2,4(1H, 3H)-dione; 5-(2-thienyl)pyrimidine-2,4(1H, 3H)-dione; 4-amino-5-(1,3-thiazol-5-yl)pyrimidin-2(1H)-one; 4-amino-5-(2-thienyl)pyrimidin-2(1H)-one; 5-(1,3-thiazol-4-yl)pyrimidine-2,4(1H, 3H)-dione; 5-(3-thienyl)pyrimidine-2,4(1H, 3H)-dione; 4-amino-5-(1,3-thiazol-4-yl)pyrimidin-2(1H)-one; or 4-amino-5-(3-thenyl)pyrimidin-2(1H)-one, or salts thereof.

In another embodiment, a compound is provided having the general formula (III):

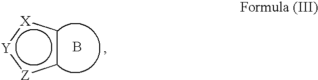

where each of X and Y is, independently, —O—, —S—, or —CH═, and X and Y are different where ever they appear, with the proviso that when X is —O— or —S—, Y is not O— or —S—, Z is selected from —CH═, —N═, or —CR¹═, and ring B is selected from:

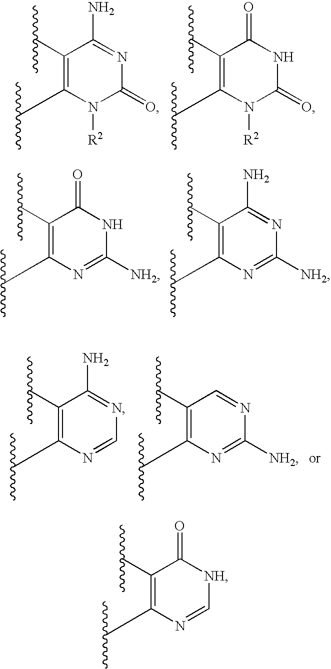

where R¹ and R² are each the same or different, where ever they appear, and each is selected from —H or a glycal having the general formula (II)a or (II)b:

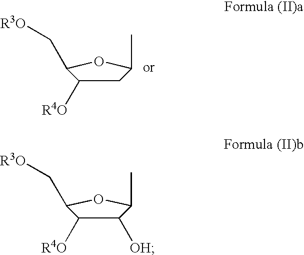

### R³ is —H, —PO₃, or:

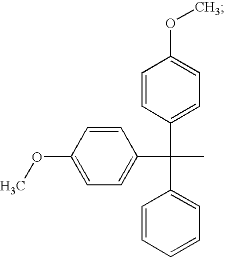

### and R⁴ is —H, —PO₃, or:

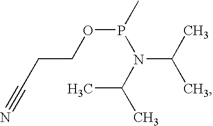

with the proviso that when R¹ is a glycal, R² is not a glycal; or salts thereof.

In one aspect, X is —O—, Z is —CR¹=, ring B is

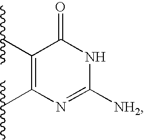

### and R¹ is

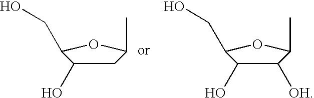

In another aspect, X is —S—, Z is —CR¹═, ring B is

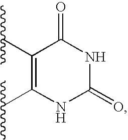

### and R¹ is

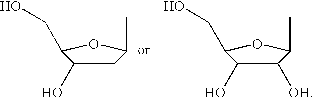

In still another aspect, X is —S—, Z is —CH═, ring B is

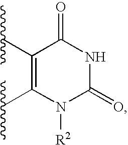

### and R² is

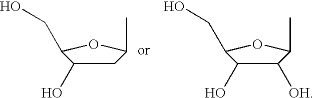

In one aspect, compounds include, but are not limited to, furo[3,2-d]pyrimidin-4-amine; 2-aminofuro[3,2-d]pyrimidin-4(3H)-one; furo[3,2-d]pyrimidine-2,4-diamine; furo[3,2-d]pyrimidin-2-amine; furo[3,4-d]pyrimidine-2,4(1H, 3H)-dione; furo[3,4-d]pyrimidin-2-amine; [1,3]oxazolo[4,5-d]pyrimidine-5,7(4H, 6H)-dione; furo[3,2-d]pyrimidine-2,4(1H, 3H)-dione; 7-amino[1,3]oxazolo[4,5-d]pyrimidin-5(4H)-one; 4-aminofuro[3,2-d]pyrimidin-2(1H)-one; furo[3,4-d]pyrimidine-2,4(1H, 3H)-dione; 4-amino[3,4-d]pyrimidin-2(1H)-one; thieno[3,2-d]pyrimidin-4-amine; 2-aminothieno[3,2-d]pyrimidin-4(3H)-one; thieno[3,2-d]pyrimidine-2,4-diamine; thieno[3,2-d]pyrimidin-2-amine; thieno[3,4-d]pyrimidine-2,4(1H, 3H)-dione; thieno[3,4-d]pyrimidin-2-amine; [1,3]thiazolo[4,5-d]pyrimidine-5,7(4H, 6H)-dione; thieno[3,2-d]pyrimidine-2,4(1H, 3H)-dione; 7-amino[1,3]thiazolo[4,5-d]pyrimidin-5(4H)-one; 4-aminothieno[3,2-d]pyrimidin-2(1H)-one; thieno[3,4-d]pyrimidine-2,4(1H, 3H)-dione; or 4-aminothieno[3,4-d]pyrimidin-2(1H)-one, or salts thereof.

In one embodiment, a compound is disclosed having the general formula (IV):

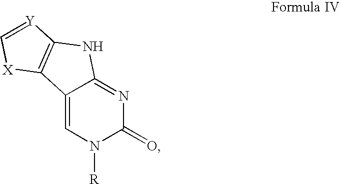

where, X and Y are, independently, —CH═, or —O—, and X and Y are different where ever they appear, with the proviso that when X is —O—, Y is not —O—,
R is —H or a glycal having the general formula (II)a or (II)b:

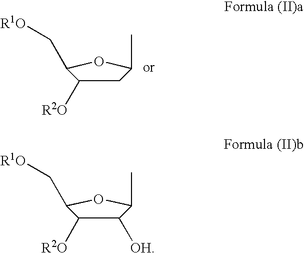

### R¹ is —H, —PO₃, or

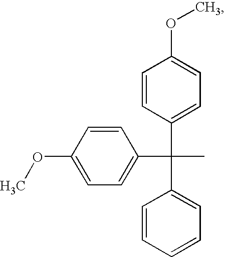

### and R² is —H, —PO₃, or

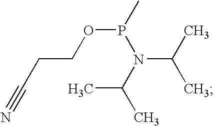

or salts thereof.

In one embodiment, a compound is disclosed having the general formula (V):

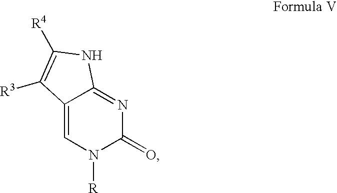

where R is —H or a glycal having the general formula (II)a or (II)b:

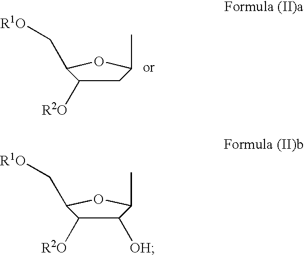

### R¹ is —H, —PO₃, or

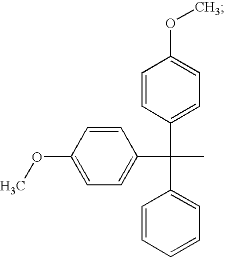

### R¹ is —H, —PO₃, or

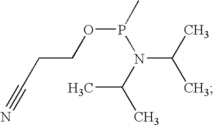

R³ and R⁴ are each independently —H or a furan having the general formula (VI):

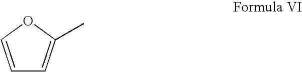

where each is different where ever they appear with the proviso that
when R³ is Formula (VI), R⁴ is not Formula (VI);
   or salts thereof.

Further, compounds of the invention include:

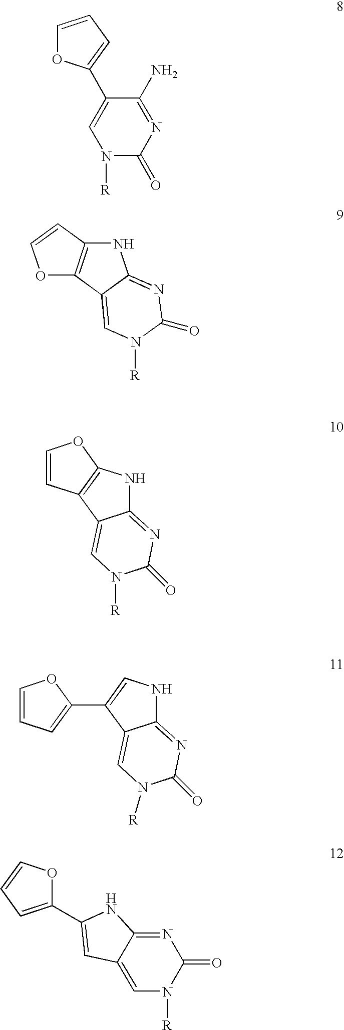

Nucleotide 8 is the dC analog of the modified T(dU) that has been synthesized by the methods disclosed herein. It is emissive (λₑₘ 443 nm φ˜1%). Nucleoside 9 is analog of 8, where the furan is fused to a new pyrrole ring (while maintaining the H-bonding capability of C). Nucleoside 10 is an isomer of 9, where the connectivity is different. Nucleosides 11 and 12 represent fused analogs of C, where a furan is conjugated but not fused to the pyrrole ring.

Further, compounds provided in the present disclosure possess a red-shifted absorption spectrum which does not substantially overlap with the absorption spectrum of a naturally occurring nucleoside, where the absorption spectrum is in the range of about 240 nm to about 350 nm, about 250 to about 320, about 262 to about 318, about 266 to about 294, about 268 to about 293, or about 286 to about 298.

In a related aspect, compounds provided in the present disclosure possess an emission spectrum in the range of about 300 to about 450, about 335 to about 435, about 337 to about 433, about 339 to about 431, or about 412 to about 413.

In one aspect, such compounds posses a long emission wavelength in the visible spectrum.

Fluorescent nucleoside analogs of the present invention are sensitive to their local environment. They can be studied using real time, sensitive assays for nucleic acids structure, dynamics and recognition. Assays measuring and detecting the fluorescent nucleoside analogs of the invention have many applications because they simplify and accelerate the accumulation of data pertinent to a specific recognition phenomenon (e.g., DNA-protein interaction, RNA-small molecule interaction). For example, in the pharmaceutical industry, such assays are essential for high throughput screening protocols, particularly in the context of drug discovery. Other applications, include studying nucleic acid modifying enzymes (e.g., DNA methyl transferases, polymerases, helicases, RNA modifying enzymes such as dicer, etc.) that play crucial roles in development, genetic diseases and cancers, the discovery of novel anti-HIV agents assisted by fluorescent TAR constructs, and the discovery of novel antibiotics targeted at the bacterial ribosome assisted by a fluorescent A-site analog, etc.

In one embodiment, a synthetic oligonucleotide is provided, including at least one compound of general formula (I) or general formula (III), where the synthetic oligonucleotide substantially hybridizes to a complementary naturally occurring polynucleotide or oligonucleotide. In a related aspect, the synthetic oligonucleotide, naturally occurring polynucleotide, and naturally occurring oligonucleotide comprise DNA or RNA.

In another embodiment, a kit is disclosed including at least one compound of the general formula (I) or general formula (II) or an oligonucleotide comprising the at least one compound, a container, and directions for using the at least one compound or oligonucleotide. In a related aspect, the at least one compound is a phosphoramidite derivative.

In one embodiment, synthetic routes are provided, according to Schemes 1-4:

Synthesis of Heterocycle:

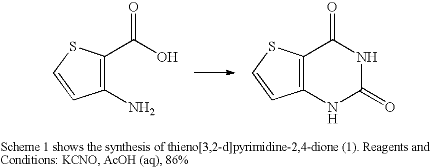

Synthesis of Ribonucleoside:

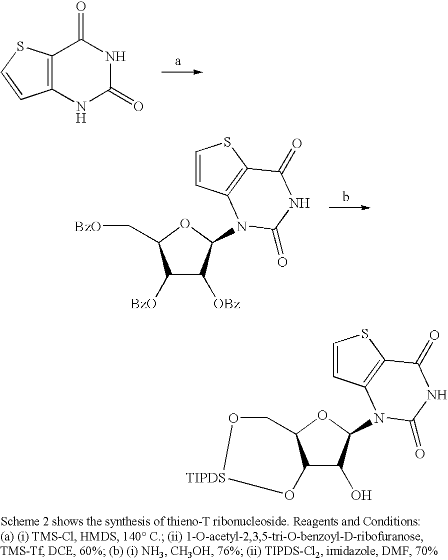

Synthesis of 2′-deoxyribonucleoside:

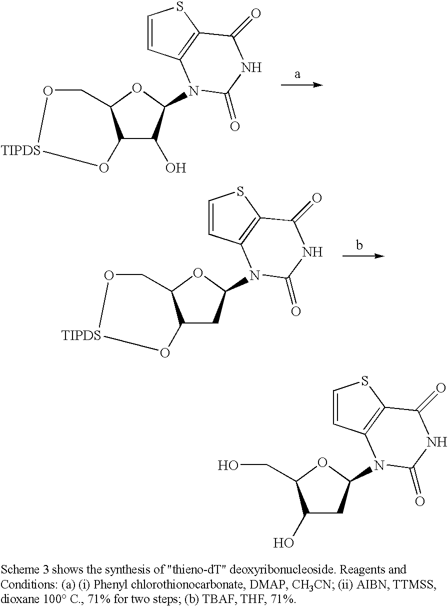

Synthesis of 2′-deoxyribonucleoside Phosphoramidite:

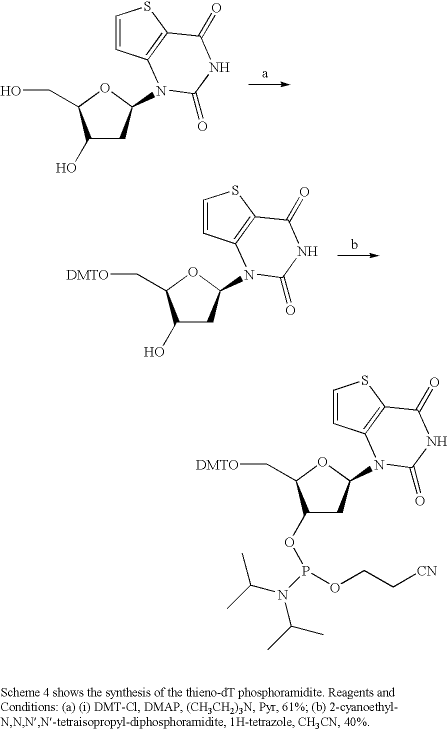

In another embodiment, synthetic routes are provided according to Schemes 5 and 6:

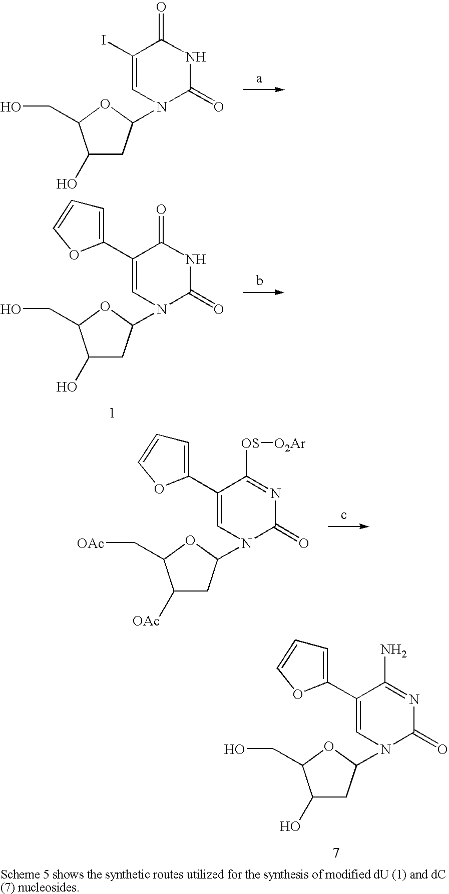

Note the use of building blocks where the fully modified dU derivative can be effectively converted into the dC analog.

Reagents: (a) 2-(Bu₃Sn)furan, PdCl₂(Ph₃P)₂, dioxane; (b) (i) Ac₂O, pyr, (ii) 2,4,6-triisopropylbenzenesulfonyl chloride, Et₃N, DMAP; (c) NH₄OH.

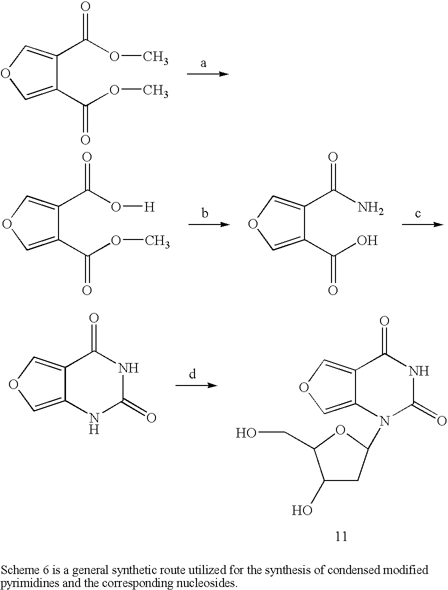

Note the last step requires separation of diastereoisomers. Reagents: (a) KOH; (b) (i) oxalyl chloride, (ii) NH₃, (iii) KOH; (c) (i) NaN₃, (ii) D; (d) (i) BSA, toluoyl protected 1-chloro-D-ribose, separation of diastereomers.

In one embodiment, a method is disclosed for synthetically preparing a fluorescently labeled oligonucleotide comprising incorporating at least one compound of the general formula (I) or general formula (III) into a DNA or RNA chain. In a related aspect, the at least one compound is admixed with a growing DNA or RNA chain, where the at least one compound is a phosphoramidite derivative. In another related aspect, such synthesis further comprises synthesis on a solid phase.

The present compositions allow for the detection of a target nucleic acid molecule, when present, in a sample. The target nucleic acid molecule can be any nucleic acid molecule that can selectively hybridize to a toehold domain of a damping oligonucleotide, particularly a damping oligonucleotide of a component of a translator. The target sequence can be a gene sequence or portion thereof (e.g., a transcriptional and/or translational regulatory sequence, coding sequence, or intron-exon junction), a cDNA molecule, an RNA (e.g., an mRNA, tRNA or rRNA), or any other nucleic acid molecule, which can be an isolated nucleic acid molecule or a nucleic acid molecule contained in a sample (e.g., a cell sample, wherein the target nucleic acid molecule is an endogenously expressed molecule or is an exogenously introduced nucleic acid molecule or expressed from an exogenously introduced molecule), and can be a naturally occurring nucleic acid molecule or a synthetic molecule. A target sequence can be any length, provided that selective hybridization with a toehold domain can occur. A target sequence also can be contained within a larger nucleic acid molecule (e.g., a restriction fragment of genomic DNA).

The sample can be any sample that can contain a nucleic acid molecule, including, for example, a biological sample, environmental sample, or chemical sample. For example, a biological sample can be a cell, tissue, or organ sample, e.g., a cell sample of an established cell line, or a tissue sample obtained from a subject (e.g., via a biopsy procedure), or a biological fluid sample, and can be a sample of eukaryotic or prokaryotic origin, including a eukaryotic cell sample that is being examined, for example, for a target sequence of an infecting microorganism. An environmental sample that can be examined for the presence (or amount) of a target nucleic acid molecule can be, for example, a forensic sample (e.g., a blood sample or hair sample from a crime scene), a water or soil sample (e.g., to identify the presence of a contaminating organism), or a washing of a solid surface (e.g., a hospital surface to be examined for the presence of an infectious organism such as an antibiotic resistant bacterium).

The compositions and methods of the invention utilize selective hybridization between a target nucleic acid molecule and a probe containing a fluorescent nucleoside analog of the present invention. Selective hybridization includes the specific interaction of a sequence of a first polynucleotide with a complementary sequence of a second polynucleotide (or a different region of the first polynucleotide). As disclosed herein, selective hybridization of a damping oligonucleotide and a propagating oligonucleotide can generate amplifier nucleic acid molecules and translators, including complexes of two oligonucleotides, three oligonucleotides, four oligonucleotides, or more. As used here, the term “selective hybridization” or “selectively hybridize” refers to an interaction of two nucleic acid molecules that occurs and is stable under moderately to highly stringent conditions. The conditions required to achieve a particular level of stringency are well known and routine, and will vary depending on the nature of the nucleic acids being hybridized, including, for example, the length, degree of complementarity, nucleotide sequence composition (for example, relative GC:AT content), and nucleic acid type, i.e., whether the oligonucleotide or the target nucleic acid sequence is DNA or RNA.

As used herein, such “conditions and time sufficient to allow for hybridization to occur” include, but are not limited to, conditions for hybridization and washing under which nucleotide sequences at least 60-70% homologous to each other typically remain hybridized to each other. The conditions can be such that sequences at least about 60%, at least about 70%, or at least about 80%, or more homologous to each other typically remain hybridized to each other. Such conditions are known to those skilled in the art and can be found in Current Protocols in Molecular Biology, John Wiley & Sons, N.Y. (1989), 6.3.1-6.3.6. One example of hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

In one embodiment, a method of detecting a target nucleic acid in a sample is provided including, but not limited to, contacting the sample with one or more oligonucleotides having at least one compound of the general formula (I) or the general formula (II) incorporated therein, for a time and under conditions sufficient to allow hybridization to occur between the target nucleic acid and the oligonucleotides, separating non-hybridized oligonucleotides, exciting the hybridized oligonucleotides, and detecting fluorescence produced by complexes formed between the oligonucleotides and the target nucleic acid, where detecting fluorescence correlates with the presence of the target nucleic acid.

In a related aspect, the target nucleic acid comprises RNA or DNA. Further, the one or more oligonucleotides may be immobilized on a solid phase or may be free in solution. Moreover, such one or more oligonucleotides may be positioned on an array.

All documents provided herein are incorporated by reference in their entirety.

The following examples are intended to illustrate but not limit the invention.

### EXAMPLE 1

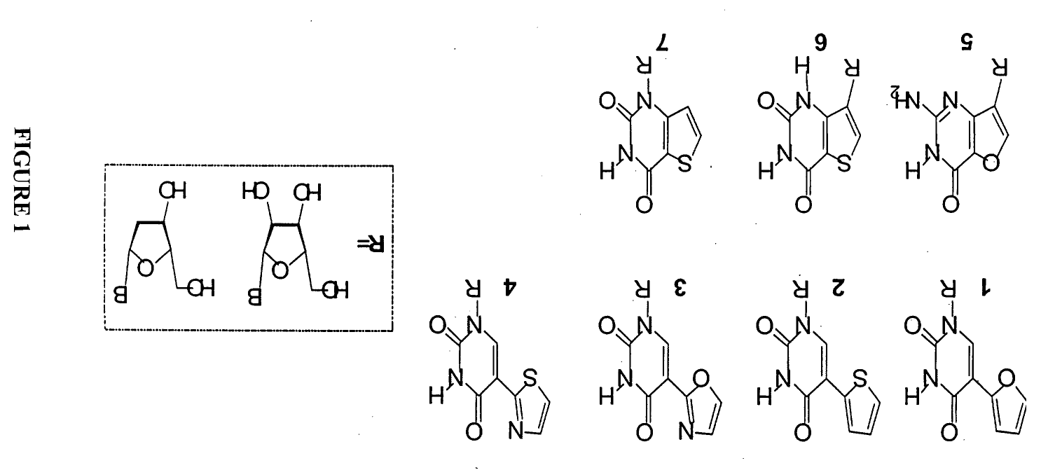

The invention methods conjugate 5-membered heterocycles to pyrimidines and purines. FIG. 1 shows fluorescent nucleoside chemical structures which are possible. The fluorescent nucleosides can be divided into subgroups. For example, Group I (A-F) are furo- and thieno-purine fluorescent analogs, Group II (G-L) are 5-modified pyrimidine fluorescent analogs, and Group III (M-P) are furo-, thieno-, and oxazolo-pyrimidine fluorescent analogs. The listing of purine and pyrimidine fluorescent analogs herein is not exhaustive and other conjugated 5-membered heterocyles synthesized is well within the scope of the present invention.

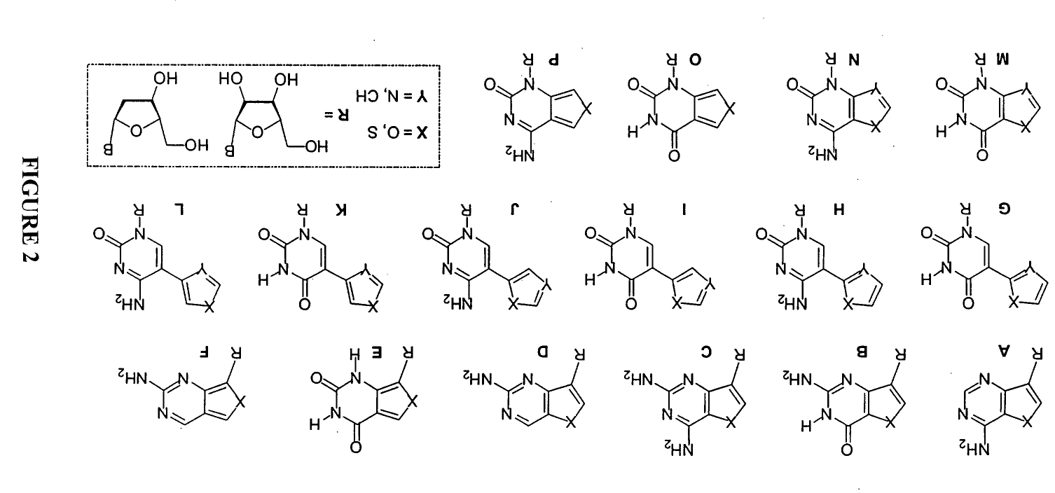

FIG. 2 shows fluorescent nucleoside structures 1-7 which have been synthesized and evaluated for their photophysical properties, including their absorption and emission spectrum. Fluorescent nucleoside structures 1-7 fluoresce and show a shift towards the red wavelength spectrum and their emission spectrum is also in a long emission wavelength. For example, fluorescent nucleoside structures 1-7 emission spectrum is in the visible wavelength range.

### EXAMPLE 2

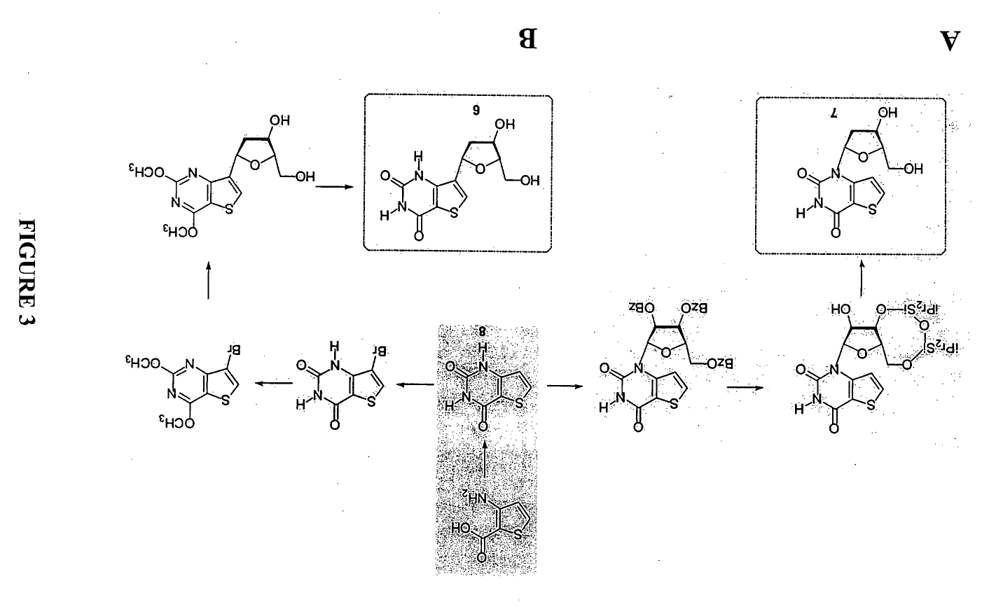

There are several methods for synthesizing fluorescent nucleoside structures of the invention. For example, fluorescent nucleoside structures 14 are prepared from halogenated pyrimidines via cross-coupling reactions. FIG. 3 shows at least two examples of fluorescent nucleoside synthesis. In one synthetic reaction, a modified N-nucleoside pyrimidine analog (e.g., structure 7) is synthesized using standard N-glycosylation. In another method, C-glycosides (e.g., structure 6) are synthesized by a cross coupling reaction using glycals and brominated heterocycles. The synthesis of structure 6 and 7 require starting material structure 8. Structure 8 is synthesized by standard transformation from simple heterocyclic precursors as shown in FIGS. 1 and 2.

### EXAMPLE 3

The fluorescent nucleoside analogs (1-7) as shown in FIG. 2 show steady state absorption and emission spectra. Table I summarizes the key photophysical parameters of select nucleosides.

TABLE I
Absorption and Emission Spectra for Modified Nucleosides in Water

| Nucleoside | Absorption maxima (nm) | Emission maxima (nm) | Φ_{F} |
|---|---|---|---|
| 1 | 250, 316 | 431 | 0.03 |
| 2 | 262, 320 | 433 | |
| 3 | 298 | 412 | |
| 4 | 262, 318 | 413 | |
| 5 | 286 | 339 | 0.01 |
| 6 | 266, 294 | 337 | |
| 7 | 268, 293 | 350 | 0.02 |

Individual absorption and emission wavelengths of fluorescent nucleoside analogs 1-7 are shown in Table I. For fluorescent nucleoside analogs 1-7, the absorption spectra range is from about 250 to about 320 nm; and the emission spectra range is from about 337 to about 433 nm. The absorption and emission spectra varies depending on various properties, including the chemical structures, for example, the R group.

For some fluorescent nucleoside analogs, time resolved experiments are performed to determine the excited state lifetime of the chromophore. For example, fluorescent nucleoside structure 1, for example, displays a 1 nano second excited state lifetime. This excited state lifetime is expected for an organic chromophore of this type. Further, quantum yield measurements demonstrate a range from about 1% to above 5%.

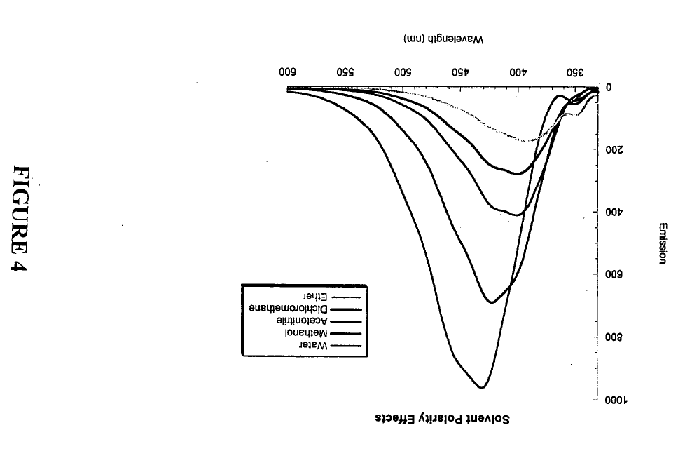

FIG. 4 shows an emission spectra for fluorescent nucleoside analog 1 in various solvents, including water (most polar), methanol, acetonitrile, dichloromethane, and diethyl ether (least polar). Fluorescent nucleoside analog 1 exhibits a hyperchromism (enhanced emission) and bathochromic (red) shift upon increasing solvent polarity. The enhanced emission and bathochromic shift is advantageous because this method distinguishes between a buried heterocycle (such as base paired and base stacked nucleobase within a perfect DNA/RNA duplex) and a solvent exposed heterocycle (upon, for example, bulging out of a nucleobase). The enhanced emission and bathochromic shift also distinguishes the fluorescent nucleoside analogs from that emission and no-shift phenomenon of naturally occurring nucleoside bases.

### EXAMPLE 4

Two fluorescent nucleoside analogs, 6 and 7, are converted into their phosphoramidites derivative and incorporated into DNA oligonucleotides using standard solid-phase DNA synthesis. The purified oligonucleotides are characterized to ensure the incorporation of the intact modified fluorescent nucleoside base. Hybridization reactions followed by thermal denaturation experiments are performed to determine structural and chemical integrity of the modified fluorescent nucleoside base. It is shown that the incorporation of the modified fluorescent nucleoside bases in the DNA oligonucleotides, form stable duplexes. Therefore, the fluorescent nucleoside analogs of the present invention are capable of hybridizing to complementary oligonucleotides similar to naturally occurring nucleoside bases.

### EXAMPLE 5

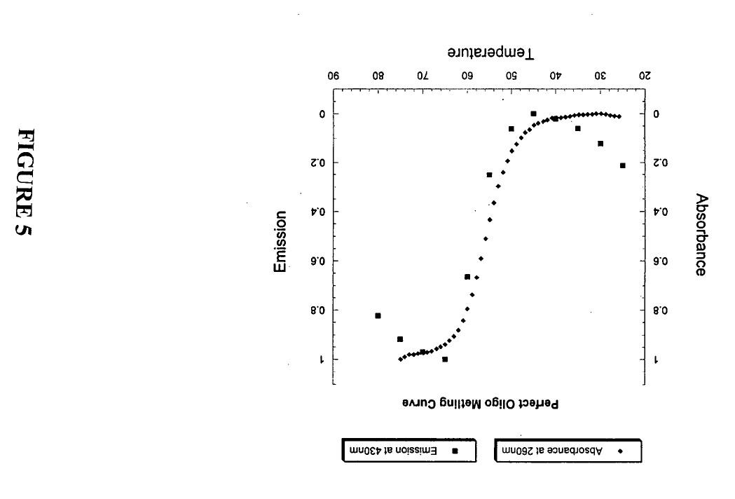

To determine the structural and chemical integrity of the fluorescent nucleoside bases, other photophysical characteristics are examined by steady state absorption and emission spectroscopy. FIG. 5, shows the sensitivity of the fluorescent nucleoside bases to their environment (e.g., absorption, emission, denaturation, etc.). These tested parameters are routinely conducted using other techniques. One advantage of this invention is that the thermal denaturation of the oligonucleotides (with incorporated fluorescent nucleoside bases) is determined by monitoring the emission spectra. FIG. 5 shows a standard thermal denaturation curve of a duplex (5′-GCG ATG 1 ATG GCG-3′) (SEQ ID NO: 1). (5′-CGC TAC A CAT CGC-3′) (SEQ ID NO: 2) containing fluorescent nucleoside structure 1 as followed by absorbance at 260 nm next to a curve determined by following the changes in fluorescence intensity of fluorescent nucleoside structure 1. As shown in FIG. 5, both curves yield approximately the same melting temperature (Tₘ=56° C.). The above result was measured in 10 mM phosphate buffer, pH 7, 100 mM NaCl, 1 HM duplex.

### EXAMPLE 6

Described below are the synthesis and photophysical characteristics of a series of simple and responsive thymidine analogs where a 2′-deoxy-U core is conjugated to aromatic 5-membered heterocycles, including furan, thiophene, oxazole and thiazole. Synthesis of nucleosides 2a-d and amidite 4 is shown in Scheme 7.

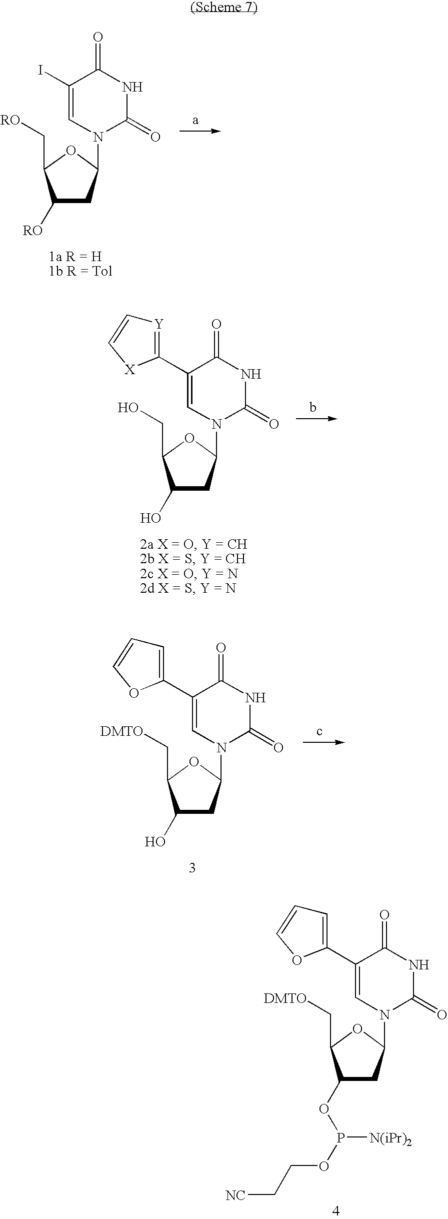

Reagents: (a) 2a: 1a, 2-(Bu3Sn)furan, PdCl2(Ph3P)2, dioxane, 94%; 2b: 1a, 2-(Bu3Sn)thiophene, PdC12(Ph3P)2, dioxane, 53%; 2c: (i) 1b, 2-(Bu3Sn)oxazole, Pd(Ph3P)4, toluene; (ii) K2CO3, 5% THF/methanol, 10%; 2d: (i) 1b, 2-(Bu3Sn)thiazole, PdC12(Ph3P)2, dioxane; (ii) K2CO3, 5% THF/methanol, 34%; (b) DMTCl, pyridine, Et3N, 71%; (c) (iPr2N)2POCH2CH2CN, 1H-tetrazole, CH3CN, 65%.

The one-step synthesis of the modified pyrimidines is straightforward (Scheme 7). It entails a palladium-mediated cross coupling of the commercially available 5-iodo-2′-deoxyuridine (or 3′,5′-diTol-Iodo-dU) and the corresponding stannylated heterocycles. Standard protection of the 5′-hydroxyl with 4,4′-dimethoxytrityl chloride followed by phosphitylation of the unprotected 3′-hydroxyl affords the building blocks necessary for solid-phase DNA synthesis (Scheme 7).

Photophysical properties of nucleosides 2a-d were examined prior to incorporation into oligonucleotides (Table 2).

TABLE 2
Photophysical data of nucleosides 2a-d

| | λₘₐₓ | λₘₐₓ | | λₑₘ | λₑₘ | |
|---|---|---|---|---|---|---|
| cm | Et₂O | H₂O | Φ | Et₂O | H₂O | I |
| pd | (nm) | (nm) | H₂O | (nm) | (nm) | H₂O/Et₂O |
| 2a | 314 | 316 | 0.03 | 395 | 431 | 5.6 |
| 2b | 320 | 314 | 0.01 | 421 | 434 | 1.6 |
| 2c | 292 | 296 | <0.01 | 390 | 400 | 1.0 |
| 2d | 318 | 316 | <0.01 | 397 | 404 | 2.1 |

5.0 × 10⁻⁵ M (λₘₐₓ), 1.0 × 10⁻⁵ M (λₑₘ), H₂O see ref 13, Φ see ref 9.

To evaluate the nucleoside's potential to respond to polarity changes, their photophysical characteristics have been evaluated in different solvents. Increasing solvent polarity has little influence on the absorption maxima of the conjugated nucleosides. In contrast, both emission wavelength and intensity are markedly affected by solvent polarity. In ether, the least polar solvent tested, nucleoside 2a, for example, displays a relatively weak emission with a maximum at 395 nm. In water, the most polar solvent examined, 2a exhibits an intense emission band which peaks around 430 nm and decays deeply into the visible (>550 nm). Solvents of intermediate polarity display an intermediate behavior with a clear emission bathochromic and hyperchromic effects with increasing solvent polarity. Nucleoside 2a, containing a conjugated furan, exhibits the highest sensitivity to solvent polarity (Table 2) and is therefore selected for incorporation into oligonucleotides.

The absorption and emission spectra of the singly modified single stranded oligonucleotide 5 are similar to those exhibited by the free nucleoside 2a in buffer. When hybridized to its perfect complement 6, a duplex (5.6) that is as stable as the control unmodified duplex 6.8 is obtained (Tm=56° C. for both). Similar to other emissive nucleosides (e.g., 2-aminopurine), the emission of the furan containing dU is significantly quenched when found in a perfectly base paired duplex. Importantly, thermal denaturation curves, determined by either absorbance at 260 nm or emission at 430 nm, yield the same melting temperature (Tm=56° C.).

| | | |
|---|---|---|
| 5 | 5′-GCG-ATG-XGT-AGC-G-3′ | (SEQ ID NO: 3) |
| 6 | 5′-CGC-TAC-ACA-TCG-C-3′ | (SEQ ID NO: 2) |
| 7 | 5′-CGC-TAC-YCA-TCG-C-3′ | (SEQ ID NO: 4) |
| 8 | 5′-GCG-ATG-TGT-AGC-G-3′ | (SEQ ID NO: 5) |

X = 2a and Y = THF residue

Abasic sites are important DNA lesions that can be generated either spontaneously or via enzymatic base excision of damaged nucleosides. Several methods have been developed for detecting the presence of these cytotoxic abasic sites, most require irreversible modifications of isolated DNA. When oligo 5 is hybridized to the tetrahydrofuran-containing oligo 7a duplex containing an abasic site is formed. Remarkably, the emission of duplex 5•7 is enhanced 7-fold when compared to the duplex 5•6, formed upon hybridization to the perfect complement. Nucleoside 2a, when incorporated into a reporter oligonucleotide, positively signals the presence of a DNA abasic site.

An unpaired base opposite an abasic site can be intrahelical or extrahelical depending on the sequence context. While not being bound by theory, the working hypothesis is that 2a is intrahelical, assuming a syn conformation. This stacked conformation protects the hydrophobic furan moiety, while projecting the hydrogen bonding face toward the major groove. Support is offered by the following: (a) duplex 5-7 is more stable than the control duplex 7•8 that contains a dT residue opposite the abasic site (Tm=39 and 35° C., respectively). The increased stability of the modified abasic duplex (ΔTₘ=+4° C.) suggests a favorable accommodation of the modified nucleobase by the duplex, and (b) the emission band observed for duplex 5•7 decays sharper (>500 nm) than when compared to the emission exhibited by the free nucleoside in solution. This is consistent with flattening of the chromophore that can be associated with the restricted rotation of the conjugated furan ring upon inclusion within the DNA duplex.

### REFERENCES

Hurley et al., Org. Lett. 2002, 4, 2305-2308.
Hawkins, Cell Biochem. Biophys., 34, 257-281 (2001).
Rist and Marino, Curr. Org. Chem. 6, 775-793 (2002).
C. A. Royer, Methods. Mol. Biol. 40, 65 (1995).
Wu and Brand, Anal. Biochem. 218, 1 (1994).
Holzwarth, Methods Enzymol. 246, 334 (1995).

All references recited are herein incorporated by reference, in their entirety. Further, although the invention has been described with reference to the above examples, it will be understood that modifications and variations are encompassed within the spirit and scope of the invention. Accordingly, the invention is limited only by the following claims.

What is claimed is:

1. A compound having the general formula (I): 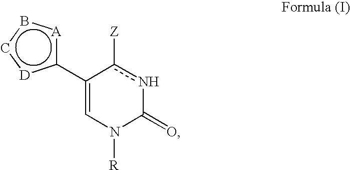
wherein dashed lines represent optional double bonds;
A and B are, independently, —CH═, —O—, or —S—, and A and B are different where ever they appear;
C and D are, independently, —N═ or —CH═; and
Z is —NH₂ or ═O,
with the proviso that when A is —O— or —S—, B is not —O— or —S—, and when C is —N═, D is not —N═;
R is —H or a glycal having the general formula (II)a or (II)b: 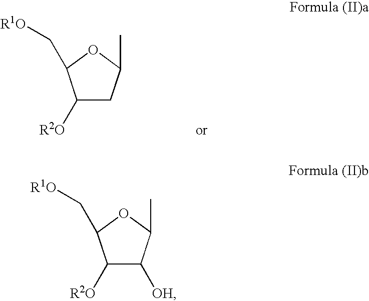
R¹ is —H, —PO₃, or 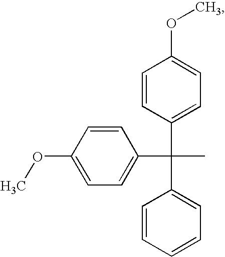
and R² is —H, —PO₃, or 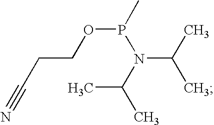
or salts thereof.

2. The compound of claim 1, wherein A is —O—, B, C, and D are —C═, Z is ═O, and R is

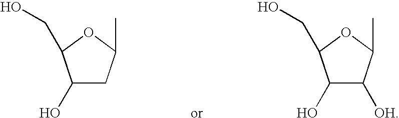

3. The compound of claim 1, wherein A is —S—, B, C, and D are —C═, Z is ═O, and R is

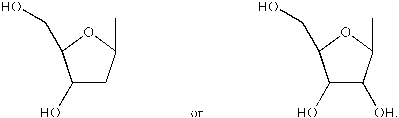

4. The compound of claim 1, wherein A is —O—, D is —N═, Z is ═O, and R is

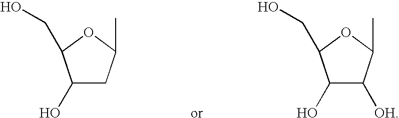

5. The compound of claim 1, wherein A is —S—, D is —N═, Z is ═O, and R is

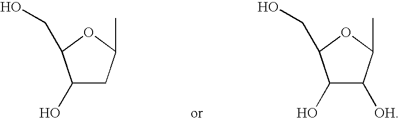

6. The compound of claim 1 selected from:
5-(1,3-oxazol-2-yl)pyrimidine-2,4(1H, 3H)-dione; 5-(2-furyl)pyrimidine-2,4(1H, 3H)-dione; 4-amino-5-(1,3-oxazol-2-yl)pyrimidin-2(1H)-one; 4-amino-5-(2-furyl)pyrimidin-2(1H)-one; 4-amino-5-(2-furyl)pyrimidin-2-(1H)-one; 5-(1,3-oxazol-5-yl)pyrimidine-2,4(1H, 3H)-dione; 5-(2-furyl)pyrimidine-2,4(1H, 3H)-dione; 4-amino-5-(1,3-oxazol-5-yl)pyrimidin-2(1H)-one; 4-amino-5-(2-furyl)pyrimidin-2(1H)-one; 5-(1,3-oxazol-4-yl)pyrimidine-2,4(1H, 3H)-dione; 5-(3-furyl)pyrimidine-2,4(1H, 3H)-dione; 4-amino-5-(1,3-oxazol-4-yl)pyrimidin-2(1H)-one; 4-amino-5-(3-furyl)pyrimidin-2(1H)-one; 5-(1,3-thiazol-2-yl)pyrimidine-2,4(1H, 3H)-dione; 5-(2-thienyl)pyrimidine-2,4(1H, 3H)-dione; 4-amino-5-(1,3-thiazol-2-yl)pyrimidin-2(1H)-one; 4-amino-5-(2-thienyl)pyrimidin-2(1H)-one; 5-(1,3-thiazol-5-yl)pyrimidine-2,4(1H, 3H)-dione; 5-(2-thienyl)pyrimidine-2,4(1H, 3H)-dione; 4-amino-5-(1,3-thiazol-5-yl)pyrimidin-2(1H)-one; 4-amino-5-(2-thienyl)pyrimidin-2(1H)-one; 5-(1,3-thiazol-4-yl)pyrimidine-2,4(1H, 3H)-dione; 5-(3-thienyl)pyrimidine-2,4(1H, 3H)-dione; 4-amino-5-(1,3-thiazol-4-yl)pyrimidin-2(1H)-one; or 4-amino-5-(3-thenyl)pyrimidin-2(1H)-one, or salts thereof.

7. The compound of claim 1, possessing a red-shifted absorption spectrum which does not substantially overlap with the absorption spectrum of a naturally occurring nucleoside.

8. The compound of claim 7, wherein the absorption spectrum is in the range of about 240 nm to about 350 nm.

9. The compound of claim 8, wherein the absorption spectrum is in the range of about 250 nm to about 320 nm.

10. The compound of claim 1, wherein the emission spectrum wavelength is in the range of about 300 nm to about 450 nm.

11. The compound of claim 10, wherein the emission spectrum wavelength is in the range of about 335 nm to about 435 nm.

12. The compound of claim 7, possessing a long emission wavelength in the visible spectrum.

13. A synthetic oligonucleotide comprising at least one compound of claim 1, wherein the synthetic oligonucleotide substantially hybridizes to a complementary naturally occurring polynucleotide or oligonucleotide.

14. The synthetic oligonucleotide of claim 13, wherein the synthetic oligonucleotide, naturally occurring polynucleotide, and naturally occurring oligonucleotide comprises DNA or RNA.

15. A compound having the general formula (III): 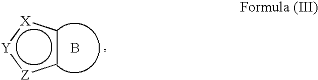
wherein each of X and Y is, independently, —O—, —S—, or —CH═, and X and Y are different where ever they appear, with the proviso that when X is —O— or —S—, Y is not —O— or —S—;
Z is selected from —CH═, —N═, or —CR¹═; and
ring B is selected from: 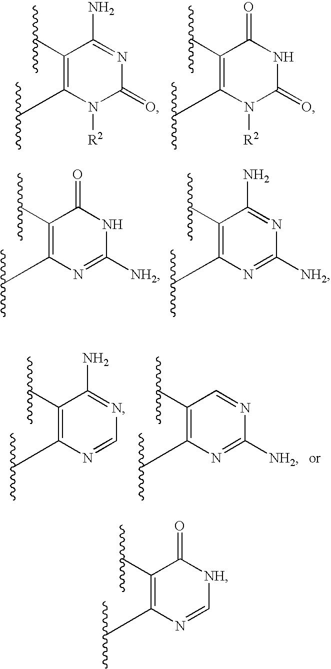
wherein R¹ and R² are each the same or different, where ever they appear, and each is selected from —H or a glycal having the general formula (II)a or (II)b: 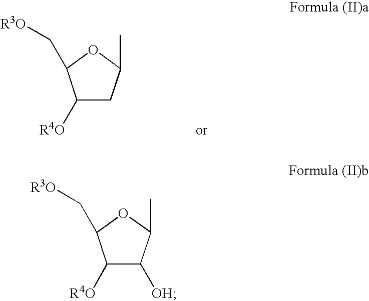
R³ is —H, —PO₃, or: 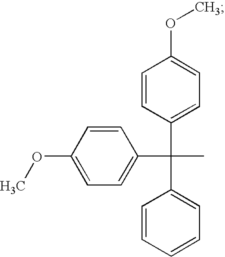
and R⁴ is —H, —PO₃, or: 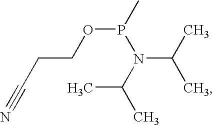
with the proviso that when R¹ is a glycal, R² is not a glycal;
or salts thereof.

16. The compound of claim 15, wherein X is —O—, Z is —CR¹═, ring B is 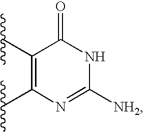
and R¹ is

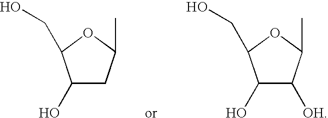

17. The compound of claim 15, wherein X is —S—, Z is —CR¹═, ring B is 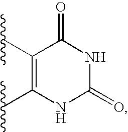
and R¹ is

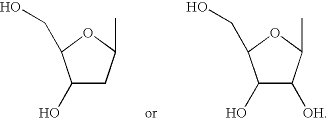

18. The compound of claim 15, wherein X is —S—, Z is —CH═, ring B is 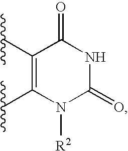
and R² is

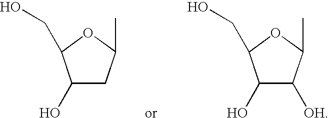

19. The compound of claim 15, selected from:
furo[3,2-d]pyrimidin-4-amine; 2-aminofuro[3,2-d]pyrimidin-4(3H)-one; furo[3,2-d]pyrimidine-2,4-diamine; furo[3,2-d]pyrimidin-2-amine; furo[3,4-d]pyrimidine-2,4(1H, 3H)-dione; furo[3,4-d]pyrimidin-2-amine; [1,3]oxazolo[4,5-d]pyrimidine-5,7(4H, 6H)-dione; furo[3,2-d]pyrimidine-2,4(1H, 3H)-dione; 7-amino[1,3]oxazolo[4,5-d]pyrimidin-5(4H)-one; 4-aminofuro[3,2-d]pyrimidin-2(1H)-one; furo[3,4-d]pyrimidine-2,4(1H, 3H)-dione; 4-amino[3,4-c]pyrimidin-2(1H)-one; thieno[3,2-d]pyrimidin-4-amine; 2-aminothieno[3,2-d]pyrimidin-4(3H)-one; thieno[3,2-d]pyrimidine-2,4-diamine; thieno[3,2-d]pyrimidin-2-amine; thieno[3,4-d]pyrimidine-2,4(1H, 3H)-dione; thieno[3,4-d]pyrimidin-2-amine; [1,3]thiazolo[4,5-d]pyrimidine-5,7(4H, 6H)-dione; thieno[3,2-d]pyrimidine-2,4(1H, 3H)-dione; 7-amino[1,3]thiazolo[4,5-d]pyrimidin-5(4H)-one; 4-aminothieno[3,2-d]pyrimidin-2(1H)-one; thieno[3,4-d]pyrimidine-2,4(1H, 3H)-dione; or 4-aminothieno[3,4-c]pyrimidin-2(1H)-one, or salts thereof.

20. The compound of claim 15, possessing a red-shifted absorption spectrum which does not substantially overlap with the absorption spectrum of a naturally occurring nucleoside.

21. The compound of claim 20, wherein the absorption spectrum is in the range of about 240 nm to about 350 nm.

22. The compound of claim 21, wherein the absorption spectrum is in the range of about 250 nm to about 320 nm.

23. The compound of claim 21, wherein the emission spectrum wavelength is in the range of about 300 nm to about 450 nm.

24. The compound of claim 23, wherein the emission spectrum wavelength is in the range of about 335 nm to about 435 nm.

25. The compound of claim 20, possessing a long emission wavelength in the visible spectrum.

26. A synthetic oligonucleotide comprising at least one compound of claim 15, wherein the synthetic oligonucleotide substantially hybridizes to a complementary naturally occurring polynucleotide or oligonucleotide.

27. The synthetic oligonucleotide of claim 26, wherein the synthetic oligonucleotide, naturally occurring polynucleotide, and naturally occurring oligonucleotide comprises DNA or RNA.

28. A kit comprising:
at least one compound of claim 1 or an oligonucleotide comprising the at least one compound;
a container; and
directions for using the at least one compound or oligonucleotide.

29. The kit of claim 28, wherein the at least one compound is a phosphoramidite derivative.

30. A kit comprising:
at least one compound of claim 15 or an oligonucleotide comprising the at least one compound;
a container; and
directions for using the at least one compound or oligonucleotide.

31. The kit of claim 30, wherein the at least one compound is a phosphoramidite derivative.

32. A method of synthesizing pyrimidine analogs comprising
a) admixing 5-iodo-2′-deoxyuridine or 3′,5′-diTol-Iodo-dU and the corresponding stannylated heterocycles in the presence of palladium;
b) protecting the 5′-hydroxyl with 4,4′-dimethoxytrityl chloride; and
c) phosphitylating the unprotected 3′-hydroxyl.

33. A method of synthetically preparing a fluorescently labeled oligonucleotide comprising incorporating at least one compound of claim 1 or 15 into a DNA or RNA chain.

34. The method of claim 33, further comprising admixing the at least one compound with a growing DNA or RNA chain, wherein the at least one compound is a phosphoramidite derivative.

35. The method of claim 34, further comprising synthesis on a solid phase.

36. A method of detecting a target nucleic acid in a sample comprising:
contacting the sample with one or more oligonucleotides having at least one compound of claim 1 or 15 incorporated therein, for a time and under conditions sufficient to allow hybridization to occur between the target nucleic acid and the oligonucleotides;
separating non-hybridized oligonucleotides;
exciting the hybridized oligonucleotides; and
detecting fluorescence produced by complexes formed between the oligonucleotides and the target nucleic acid,
wherein detecting fluorescence correlates with the presence of the target nucleic acid.

37. The method of claim 36, wherein the target nucleic acid comprises RNA or DNA.

38. The method of claim 36, wherein the one or more oligonucleotides are immobilized on a solid phase.

39. The method of claim 38, wherein the one or more oligonucleotides are positioned on an array.

40. A compound having the general formula (IV): 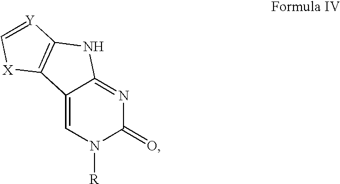
where, X and Y are, independently, —CH═, or —O—, and X and Y are different where ever they appear, with the proviso that when A is —O—, B is not —O—,
R is —H or a glycal having the general formula (II)a or (II)b: 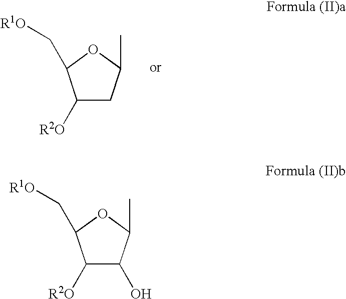
R¹ is —H, —PO₃, or 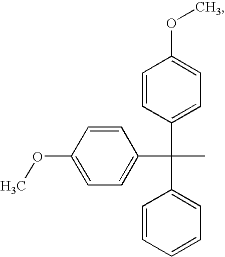
and R² is —H, —PO₃, or 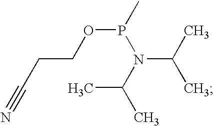
or salts thereof.

41. A compound having the general formula (V): 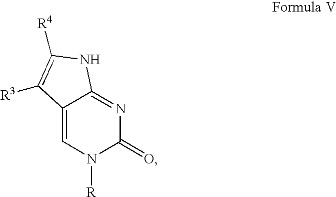
wherein R is —H or a glycal having the general formula (II)a or (II)b: 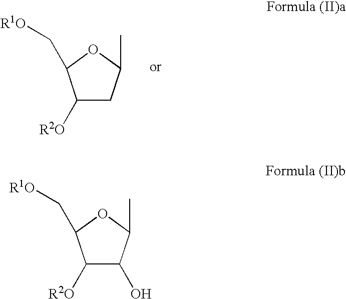
R¹ is —H, —PO₃, or 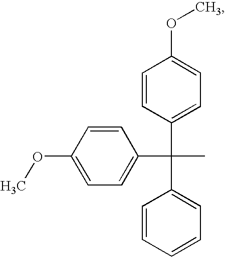
R² is —H, —PO₃, or 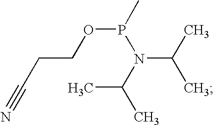
R³ and R⁴ are each independently —H or a furan having the general formula (VI): 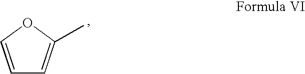
wherein each R³ and R⁴ is different where ever they appear with the proviso that when R³ is Formula (VI), R⁴ is not Formula (VI);
or salts thereof.


SEQUENCE LISTING (5 sequences)

SEQ ID NO: 1 - DNA, 13 bp, Artificial sequence
  Synthetic construct
gcgatgnatggcg

SEQ ID NO: 2 - DNA, 13 bp, Artificial sequence
  Synthetic construct
cgctacacatcgc

SEQ ID NO: 3 - DNA, 13 bp, Artificial sequence
  Synthetic construct
gcgatgngtagcg

SEQ ID NO: 4 - DNA, 13 bp, Artificial sequence
  Synthetic construct
cgctacncatcgc

SEQ ID NO: 5 - DNA, 13 bp, Artificial sequence
  Synthetic construct
gcgatgtgtagcg